(19) United States
(12) Patent Application Publication
(10) Pub. No.: US 2011/0033519 A1
(43) Pub. Date: Feb. 10, 2011

(54) Device with Aromatase Inhibitor for the Treatment and Prevention of Uterine Fibroids and Method of Use

(76) Inventor: Madeline LEONG, Chevy Chase, MD (US)

Correspondence Address: ROTHWELL, FIGG, ERNST & MANBECK, P.C., 1425 K STREET, N.W., SUITE 800, WASHINGTON, DC, 20005, US

(21) Appl. No.: 12/851,886

(22) Filed: Aug. 6, 2010

Related U.S. Application Data
(60) Provisional application No. 61/232,239, filed on Aug. 7, 2009.

(51) Int. Cl. A61F 6/14; A61K 31/566; A61K 31/4196; A61K 31/451; A61K 31/4545; A61K 31/437; A61K 31/352; A61P 35/00; A61P 15/18

(52) U.S. Cl. 424/432; 514/177; 514/383; 514/328; 514/318; 514/179; 514/300; 514/453

(57) ABSTRACT

The invention provides a device and a compound for the prevention and/or treatment of uterine fibroids in human patients, wherein the compound is dispensed from the device and is administered locally to the uterine tissue.

### CROSS REFERENCE TO RELATED APPLICATION

This application is a non-provisional application which claims the benefit of provisional application U.S. Ser. No. 61/232,239, filed Aug. 7, 2009, which is incorporated herein by reference in its entirety.

### BACKGROUND OF THE INVENTION

1. Technical Field

This invention generally relates to the fields of medical gynecology and gynecological surgery, and to conditions with benign tumors. This application describes and claims a device, one embodiment of which is an intrauterine device (IUD), together with a composition which is dispensed from the device. The composition can be administered locally to the uterus of a human patient and can be used to prevent and/or treat uterine fibroids. The device can also contain and dispense additional compounds, such as contraceptives.

2. Description of the Background Art

Women today have a 70% lifetime risk of uterine fibroids, non-cancerous tumors that cause heavy menstrual bleeding, pelvic pain and pressure, and infertility. Fibroids represent a serious public health burden with an annual treatment cost of over $2 billion in the U.S. Over 200,000 women undergo hysterectomies for fibroid-derived symptoms every year. An abdominal hysterectomy costs approximately $20,000 and requires six weeks of recovery time. To date, there is no long-term medical therapy for the treatment or prevention of fibroids.

Fibroids are benign smooth muscle tumors of the uterus found in 70-80% of women by age 50. African-American women have a three-fold higher incidence of clinically recognized fibroids than women of other races, when measured in women post-myomectomy. Depending on size and location, fibroids may cause the following: bleeding symptoms such as menorrhagia and metrorrhagia; pressure symptoms such as bloatedness, urinary frequency, and bowel disturbance; pelvic pain and dysmenorrhea; and compromised reproductive function, which may lead to early pregnancy loss and later pregnancy complications.

Fibroids are mainly treated by surgery, either hysterectomy or myomectomy. While surgery is appropriate for some women, it is high risk for others, especially those with comorbidities like obesity and heart disease. Furthermore, surgical options have significant drawbacks. For instance, abdominal hysterectomy has a six-week recovery time, frequent (30%) minor complications, and a mortality rate of 0.38-1 per 1000 patients. Hysterectomy is also associated with earlier menopause and severe psychosocial stress. Fibroids are the primary indication (32%) for hysterectomy and result in an additional 30,000 myomectomies per year (U.S. data). Two other costly, but less invasive, procedures are performed for fibroids: uterine artery embolization (UAE) and magnetic-resonance guided focused ultrasound (MRgFUS). The estimated cost of hysterectomy is $20,000 per patient. Other treatments are more expensive: Myomectomy costs $35,000 per patient, UAE $29,000, and MRgFUS $27,000. Based on U.S. insurance data, the annual cost to a woman with symptomatic fibroids is $4,600, $800 of which is due to lost work. For comparison, the five-year cost of an IUD is $600 to $900 per patient.

Currently, the only FDA-approved non-surgical drug therapy for fibroids is the administration of gonadotropin-releasing hormone (GnRH) agonists, which shrink fibroids but also cause hot flashes and other symptoms of menopause. Use of GnRH agonists for more than six months can result in irreversible bone loss. Because of this, GnRH agonists do not provide safe, long-term treatment of fibroids.

A newer option for medical management of fibroids is the oral administration of aromatase inhibitors (AIs), including Letrozole, Anastrozole, and Exemestane. Exemestane is a steroidal AI, while Anastrozole and Letrozole are non-steroidal AIs. A recent study of 35 women showed a 45% reduction in fibroid volume after treatment with Letrozole for three months (2.5 mg per day orally). Another trial of 16 women reported a 47% reduction in fibroid volume after three months of oral Letrozole at a dose of 5 mg/day. Similarly, three months of oral Anastrozole at a dose of 1 mg/day produced a 56% reduction in fibroid volume. Unfortunately, oral administration of AIs cause significant osteoporosis like GnRH agonists.

Accordingly, there is a great need in the art for a method of treating and/or preventing fibroids to avoid the unwanted and serious adverse effects of the traditional surgical and oral treatments discussed above. In particular, there is a need in the art for a treatment that can be administered long-term to a patient while avoiding the adverse effect of causing osteoporosis. Such treatments also preferably include the administration of additional compounds which human patients frequently require, including contraceptives.

### SUMMARY OF THE INVENTION

As discussed above, oral administration of AIs cause significant osteoporosis, as do GnRH agonists. However, there is a key difference between the two drug classes: GnRH agonists act on the pituitary gland, while AIs act locally in the uterus by blocking the conversion of androgens to estrogen. Because the mechanism of AIs does not require downregulation of the hypothalamic-pituitary axis, these drugs can be effectively targeted to a specific organ.

Accordingly, an embodiment of the invention provides a method comprising delivery of at least one AI locally to the uterus. The method can include an extended or controlled release aspect to the delivery rate, as well. The method also optionally includes the local co-administration of an additional agent, for example, a contraceptive.

An additional embodiment of the invention provides a method comprising treating fibroids in a patient by delivering at least one AI locally to the uterus. The method can include an extended or controlled release aspect to the delivery rate, as well. The method also optionally includes the local co-administration of an additional agent, for example a contraceptive. This method can be used to treat symptomatic or asymptomatic fibroids, to treat fibroids prior to surgery or following surgery, and to treat fibroids that have recurred after surgery.

An additional embodiment of the invention provides a method comprising preventing fibroids in a patient by delivering an AI locally to the uterus. The method can include an extended or controlled release aspect to the delivery rate, as well. The method also optionally includes the local co-administration of an additional agent, for example a contraceptive. This method can be used to in a patient prior to the development of fibroids, or post-surgery to prevent recurrence of fibroids.

An additional embodiment of the invention provides a device for delivering an AI locally to the uterus. In one embodiment, the device can be an IUD. In another embodiment, the device can be another standard gynecological device, such as a vaginal ring or gel. The device can also be compatible with an extended or controlled release formulation of the compound, and optionally can also co-administer an additional agent, for example a contraceptive.

An additional embodiment of the invention provides a formulation comprising an active compound which blocks the activity of the aromatase enzyme or otherwise affects the hormonal balance present in the uterus, and optionally, the active compound is administered together with active agents, or inert agents which provide an extended or controlled release functionality. The formulation may optionally contain additional active agents, such as contraceptives. The additional active agents may be mixed in the same formulation with the active compounds or may be in a separate formulation. In the latter case, the formulation of the active compound is chemically compatible with the separate formulation of the additional active agents. In one embodiment, the active compound is the steroid Exemestane. In one embodiment, the IUD contains a reservoir for storing and dispensing the AI. The additional active agents may also be stored and dispensed from the reservoir containing the AI, or may be stored and dispensed from a separate reservoir.

In another embodiment, the active compound is an AI described herein. In one embodiment, the contraceptive is preferably copper ions. The formulation also optionally contains additional active agents or inert ingredients. Preferred formulations contain about 0.0001% to about 99% of active compound by weight, about 5% to about 95% of active compound by weight, about 0.001% to about 25% of active compound by weight, or about 0.01% to about 20% of active compound by weight.

### BRIEF DESCRIPTION OF THE FIGURE DESCRIPTIONS

The accompanying drawings, which are incorporated herein and form part of the specification, illustrate various embodiments of the present invention.

FIG. 1: Aromatase-inhibitor loaded IUD in accordance with an embodiment of the present invention.

FIG. 2: Aromatase-inhibitor loaded IUD in accordance with another embodiment.

FIG. 3: Aromatase-inhibtor loaded IUD in accordance with one embodiment of the present invention positioned in situ.

FIGS. 4A, 4B and 4C: Aromatase inhibitor-loaded IUDs in accordance with further embodiments of the present invention.

### DETAILED DESCRIPTION OF THE PREFERRED EMBODIMENTS

The present invention has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

Fibroids typically grow during women's reproductive years and shrink after menopause. The primary risk factors for fibroids, including early age of menarche and obesity, result in greater exposure to endogenous estrogen.

The natural history of these tumors is highly variable: According to a growth study of 262 fibroids in premenopausal women, the median growth rate was 9% per six months. However, the range in growth rate was −89% to +138%, and fibroids from the same woman grew at different rates.

This variability makes it difficult to counsel young women with small fibroids. Clinically “silent” fibroids may or may not grow rapidly to cause severe symptoms. Currently, most physicians advise “watchful waiting” for women with asymptomatic fibroids, but this may be the optimal time for an intervention to prevent further growth.

Because fibroids are estrogen-dependent, uterine tissue continually exposed to AIs should become an unfavorable milieu for fibroid growth and formation—thus preventing, slowing or treating the disease. According to data from breast cancer trials, long-term use of AIs for multiple years does not cause any adverse effects to the uterus.

Recent biochemical studies show AIs induce apoptosis of fibroid cells. The level of aromatase mRNA is 1.5-25 times higher in fibroids than in the surrounding myometrium. This data, as well as the previously noted clinical trials, indicate that localized delivery of AIs should be considered as a treatment for the primary prevention of fibroids.

Fibroids are classified as within the wall of the uterus (intramural), inside the uterine cavity (intra-cavitary), or attached to the outer uterine wall (subserosal or pedunculated). Submucosal fibroids lie within or close to the uterine cavity while subserosal fibroids impinge on the uterine serosa. Still other fibroids, called pedunculated, are attached to the uterus by a stalk.

Intramural fibroids would be best treated by the present invention. It is uncertain whether pedunculated or intra-cavitary fibroids would receive enough drug to cause shrinkage. Intra-cavitary fibroids can be removed by hysteroscopic resection, and pedunculated fibroids by laparoscopic myomectomy. More invasive procedures like hysterectomy and abdominal myomectomy are often required for large intramural fibroids, which would be targeted by the present invention. Despite these limitations, the treatment discussed herein should dramatically decrease fibroid incidence.

An embodiment of the present invention is directed to an intra-uterine delivery of at least one AI, which is known to treat and prevent fibroids. One advantage of this targeted drug delivery is the elimination of systemic adverse effects, including osteoporosis. This allows safe, long-term use of the therapy. An additional embodiment of the invention is a novel device which treats and/or prevents fibroids, while simultaneously providing contraception. The present invention is described below with reference to the device being an IUD. However, it is understood that any device which is capable of delivery the active ingredients to the uterus can be used, including those devices described herein.

First used more than 30 years ago, IUDs capable of targeted drug delivery to the uterus are well known in the art. For example, the Mirena® IUD (Leiras-Schering), marketed in 2001, releases a constant daily dose of the steroid progesterone for five years (20 μg/day levonorgestrel), which provides contraception. Another IUD used for contraception is Paraguard® (Duramed). Paraguard, also known as the Copper T 380A, prevents pregnancy by releasing copper ions, which impair sperm motility and viability. Ninety-five percent of human sperm incubated in a solution of copper ions is immotile after 120 minutes. Because progesterone may stimulate fibroid growth, it is preferable to use copper sleeves to provide contraception for the AI-loaded IUD.

Another steroid packaged in an IUD is Danazol, studied for the treatment of endometriosis. In one study, an IUD was loaded with 400 mg Danazol for 6 months of treatment. The following US patents and patent applications, each incorporated herein by reference, also generally describe how to construct an IUD which is capable of drug delivery to the uterus: U.S. Pat. Nos. 7,528,145, 6,537,566, 6,083,916, 2006/0106076, 2006/0264912, 2008/0096950 and 2009/0123522.

IUDs that are contemplated for use in this invention include framed and frameless. Framed IUDs, such as Mirena® and Paraguard®, have a plastic upper portion shaped like a T, which is designed to prevent expulsion of the device. Frameless IUDs, such as GyneFix®, are directly attached to the uterine myometrium using an anchoring knot of suture. Clinical trials indicate frameless devices may reduce the risk of expulsion, abnormal bleeding, and pain encountered with framed devices. A frameless device may be particularly appropriate for the uterine cavity distorted by intramural or submucosal fibroids.

AIs are a broad class of pharmacologic agents that inhibit aromatase, an enzyme which converts androgens into estrogens. Aromatase inhibitors then prevent the formation of estrogens, thus decreasing the amount available to act on tissues with estrogen receptors, including fibroids. AIs can have a steroid structure, such as Exemestane. These AIs act by irreversibly binding aromatase, thus preventing the androgen to estrogen conversion. AIs can also have a non-steriod structure, such as Anastrozole and Letrozole. These AIs act by reversibly inhibiting aromatase, thus preventing the androgen to estrogen conversion. AIs have been traditionally used to treat breast cancer. Although widespread in use, major side effects of systemic treatment with AIs include joint disorders, osteoporosis which can lead to fractures, and hypercholesterolemia.

AI compounds that are contemplated for use with the invention include Exemestane (Aromasin®), Anastrozole (Arimidex®), Letrozole (Femara®), Aminoglutethimide (Cytadren®), Rogletimide (an analog of aminoglutethimide), Formestane (Lentaron®), Fadrozole (Afema®), Testolactone (Teslac®), Vorozole (Rivizor®), 4-androstene-3,6,17-trione (“6-OXO”), 1,4,6-androstatrien-3,17-dione (ATD), and 4-hydroxyandrostenedione (4-OHA), or any combination thereof. The following US patent applications, each incorporated herein by reference, also generally describe compounds which could be used with the present invention: 2006/0030570 and 2008/0051329.

Contraceptive compounds that are contemplated for use with the invention include, for example, cupric ions, gold ions, silver ions, copper and polymer nanoparticles, and copper-containing composites (e.g., polyvinyl alcohol containing cupric ions) or any combination thereof.

In addition to treating and preventing fibroids, the devices, methods, formulations and compounds of the present invention are contemplated for use with additional tumor diseases, including adenomyosis and endometriosis.

Appropriate doses of the contraceptive controlled release formulation will vary according to the contraceptive chosen for a particular device. Construction of such devices and selection of appropriate contraceptives are well known in the art. Pharmaceutical compositions containing a compound of the present invention as the active ingredient can be prepared according to conventional pharmaceutical compounding techniques. See, for example, Remington: The Science and Practice of Pharmacy, 21st Ed., Lippincott Williams & Wilkins, Philadelphia, 2005. Typically, an antagonistic amount of active ingredient will be admixed with a pharmaceutically acceptable carrier. “Pharmaceutical composition” means physically discrete coherent portions suitable for medical administration. As used herein, the term “pharmaceutically acceptable” carrier means a non-toxic, inert solid, semi-solid liquid filler, microparticle, nanopaticle, powder, suspension, solution, film, diluent, encapsulating material, formulation auxiliary of any type.

The active agent is preferably administered in a therapeutically effective amount. By a “therapeutically effective amount” or simply “effective amount” of an active compound is meant a sufficient amount of the compound to treat the desired condition at a reasonable benefit/risk ratio applicable to any medical treatment. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy. The active agent is preferably administered in an therapeutically effective amount.

Appropriate doses of some examples of a AI controlled release formulation are described in Table 1. The IUD of the present invention is contemplated for use over a five to ten year period. The formulation of the AI is likewise contemplated for use over this same time period and as such is designed to permit a steady release of AI over that period of time. The AI formulation used in the contemplated device is stable at physiological temperatures for this same period of time.

TABLE 1

| Aromatase inhibitor | Possible dosage delivered by IUD per day⁴ |
|---|---|
| Testolactone | 1 μg-2000 mg |
| Aminoglutethimide | 1 μg-1000 mg |
| Rogletimide | 1 μg-1000 mg |
| Formestane | 1 μg-500 mg |
| Exemestane | 1 μg-50 mg |
| Letrozole | 1 μg-20 mg |
| Anastrozole | 1 μg-10 mg |
| Fadrozole | 1 μg-10 mg |
| Vorozole | 1 μg-10 mg |

The pharmaceutical compositions will generally contain from about 0.0001 to 99 wt. %, preferably about 0.001 to 50 wt. %, more preferably about 0.01 to 10 wt. % of the active ingredient by weight of the total composition. In addition to the active agent, the pharmaceutical compositions and medicaments can also contain other pharmaceutically active compounds, such as contraceptives described herein. When used with other pharmaceutically active compounds, the AIs of the present invention may be delivered in the form of drug cocktails. A cocktail is a mixture of any one of the compounds useful with this invention with another drug or agent. In this embodiment, a common administration vehicle would contain both the instant composition in combination with a supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

### EXAMPLES

The present invention can be described by reference to the following Examples, which are offered by way of illustration and are not intended to limit the invention in any manner. Standard techniques well known in the art or the techniques specifically described below were utilized.

### Example 1

An embodiment of the invention consists of a non-biodegradable 00-monofilament polypropylene suture attached to a membrane-controlled reservoir system (MCRS) and to copper sleeves. The total surface area of the copper sleeves should be approximately 330 mm. An anchoring knot will allow implantation of the frameless IUD into the uterine cavity which is surrounded by the uterine fundus.

The MCRS consists of an AI and ethylene vinyl acetate (EVA). This combination can be cast into a mold and freeze-dried until solid to provide timed-release localized drug delivery. The permeability of the EVA copolymer membrane can be modified by adjusting the vinyl acetate content.

Release of the drug can be evaluated in vitro by high performance liquid chromatography (HPLC) and should show zero-order kinetics.

Since the market for the proposed IUD includes many reproductive-age women, it is essential that the device be supplied only to reproductive-age women using contraception at the time. If a woman becomes pregnant with an IUD in place, miscarriage is likely. IUDs are effective, easy to use, and last for five to ten years. They can be removed immediately to allow pregnancy or for any other reason.

### Example 2

Ethylene vinyl acetate (EVA) is dissolved in an organic solvent such as methylene chloride. An aromatase inhibitor in solid form is mixed with an inert sugar and added to the liquid solution of EVA. This combination will be cast into a mold and freeze-dried until solid to create the membrane-controlled reservoir system (MCRS). The permeability of the EVA copolymer membrane can be modified by adjusting the vinyl acetate content.

The lower portion of the MCRS is preferably attached to a non-biodegradable monofilament suture, which will extend through the cervix to allow removal of the device. The top portion of the MCRS is attached to either a plastic frame or additional suture to create a framed or frameless IUD, respectively. The framed IUD will have expandable arms to prevent expulsion while the anchoring knot of the frameless IUD will be implanted directly into the uterine fundus.

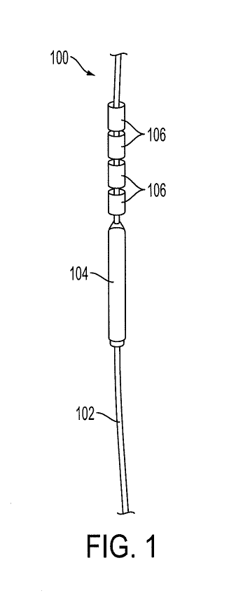

FIG. 1 shows a diagram of an IUD 100 constructed out of non-biodegradable suture material 102 with a controlled release reservoir 104 containing an AI arranged on the longitudinal axis. Also on the longitudinal axis, are four copper sleeves 106 arranged end-to-end in series fashion. In an embodiment, the copper sleeves 106 are 5 mm long and 2.2 mm in diameter.

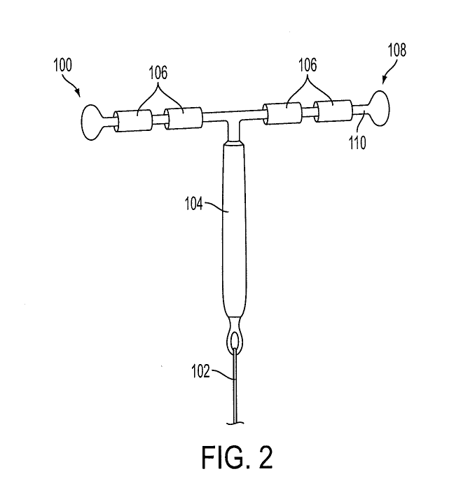

FIG. 2 shows a diagram of an IUD 100 according to another embodiment. The IUD 200 is constructed out of non-biodegradable suture material 102 with a controlled release reservoir 104 containing an AI arranged in the longitudinal axis. However, this IUD 200 includes a frame 108 with a transverse cross member 110 perpendicular to the longitudinal axis of the reservoir 104. Also, on the transverse axis, are four copper sleeves 106, which are arranged serially to one another and perpendicular to the controlled release reservoir 104. Two copper sleeves 106 are shown on each arm of the transverse cross member 110.

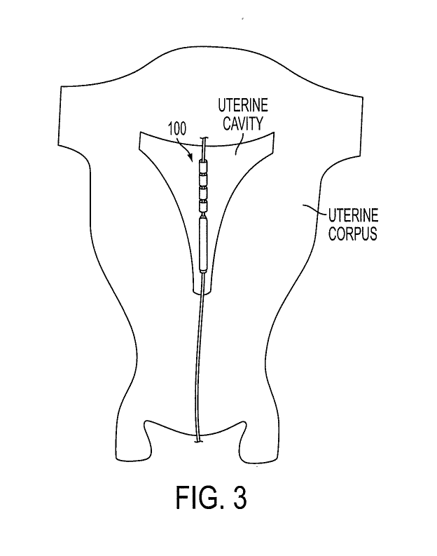

FIG. 3 shows a diagram of the device 100 of FIG. 1, showing the placement of the IUD 100 in the uterine cavity surrounded by the uterine corpus.

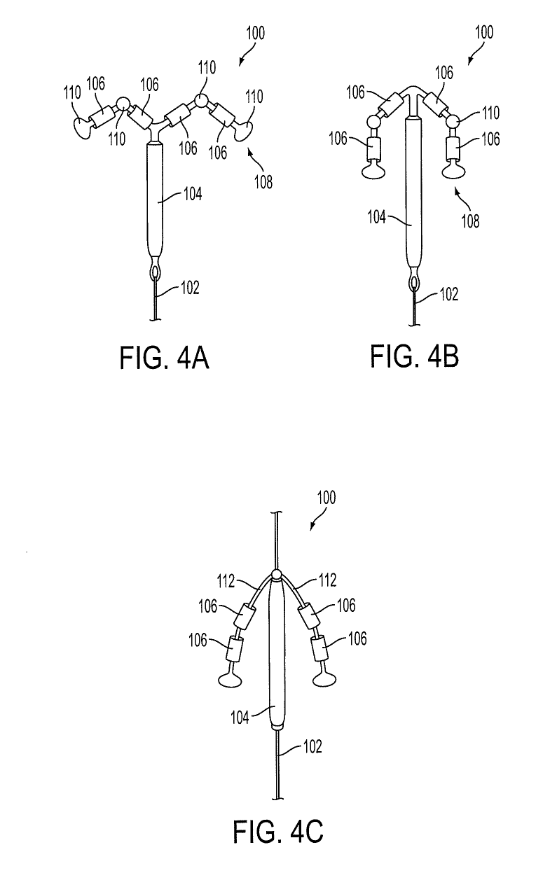

FIGS. 4A, 4B and 4C each show alternative embodiments of an IUD 100 with a controlled release reservoir 104 containing an AI arranged on the longitudinal axis, and additionally show the IUD containing four copper tubes 106 arranged on the transverse axis. The IUDs 100 in FIGS. 4A and 4B include frames 108 with differently shaped arms 110 made up of segmented portions with a copper tube 106 on each segment. The IUD 100 in FIG. 4C is frameless, with the copper tubes 106 supported on a pair of thread-like appendages 112 made of non-biodegradable suture material, which allows the IUD to reside in unusually shaped uterine cavities, including those distorted by fibroids.

In addition, at least four copper sleeves 5 mm long×2.2 mm diameter are preferably attached to the IUD to provide contraception. The total surface area of the copper sleeves should be approximately 330 mm.

The IUD is preferably packaged in a plastic tubular inserter. It will be inserted into the uterus using standard equipment, as necessary, including a speculum, uterine sound, and uterine clamp.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

The references listed below, and all references cited in the specification are hereby incorporated by reference in their entirety.
1. Day Baird D, Dunson D B, Hill M C, Cousins D, Schectman J M. High cumulative incidence of uterine leiomyoma in black and white women: ultrasound evidence. Am J Obstet Gynecol 2003; 188:100-7.
2. Zowall H, Cairns J A, Brewer C, Lamping D L, Gedroyc W M, Regan L. Cost-effectiveness of magnetic resonance-guided focused ultrasound surgery for treatment of uterine fibroids. BJOG 2008; 115:653-62.
3. Farquhar C M, Harvey S A, Yu Y, Sadler L, Stewart A W. A prospective study of 3 years of outcomes after hysterectomy with and without oophorectomy. Am J Obstet Gynecol 2006; 194:711-7.
4. Parker W H. Etiology, symptomatology, and diagnosis of uterine myomas. Fertil Steril 2007; 87:725-36.
5. O'Sullivan A K, Thompson D, Chu P, Lee D W, Stewart E A, Weinstein M C. Cost-effectiveness of magnetic resonance guided focused ultrasound for the treatment of uterine fibroids. Int J Technol Assess Health Care 2009; 25:14-25.
6. Hartmann K E, Birnbaum H, Ben-Hamadi R, et al. Annual costs associated with diagnosis of uterine leiomyomata. Obstet Gynecol 2006; 108:930-7.
7. Trussell J, Lalla A M, Doan Q V, Reyes E, Pinto L, Gricar J. Cost effectiveness of contraceptives in the United States. Contraception 2009; 79:5-14.
8. Hornstein M D, Surrey E S, Weisberg G W, Casino L A. Leuprolide acetate depot and hormonal add-back in endometriosis: a 12-month study. Lupron Add-Back Study Group. Obstet Gynecol 1998; 91:16-24.
9. Parsanezhad M E, Azmoon M, Alborzi S, et al. A randomized, controlled clinical trial comparing the effects of aromatase inhibitor (letrozole) and gonadotropin-releasing hormone agonist (triptorelin) on uterine leiomyoma volume and hormonal status. Fertil Steril 2009.
10. Gurates B, Parmaksiz C, Kilic G, Celik H, Kumru S, Simsek M. Treatment of symptomatic uterine leiomyoma with letrozole. Reprod Biomed Online 2008; 17:569-74.
11. Varelas F K, Papanicolaou A N, Vavatsi-Christaki N, Makedos G A, Vlassis G D. The effect of anastrazole on symptomatic uterine leiomyomata. Obstet Gynecol 2007; 110:643-9.
12. Thiery M. Intrauterine contraception: from silver ring to intrauterine contraceptive implant. Eur J Obstet Gynecol Reprod Biol 2000; 90:145-52.
13. Cobellis L, Razzi S, Fava A, Severi F M, Igarashi M, Petraglia F. A danazol-loaded intrauterine device decreases dysmenorrhea, pelvic pain, and dyspareunia associated with endometriosis. Fertil Steril 2004; 82:239-40.
14. Valenzano Menada M, Costantini S, Moioli M, et al. Evaluation of endometrial thickness in hormone receptor positive early stage breast cancer postmenopausal women switching from adjuvant tamoxifen treatment to anastrozole. Breast 2008; 17:631-6.
15. Han M, Kim J Y, Park J E, Kim J M, Lee K S. Effects of letrozole on proliferation and apoptosis in cultured leiomyoma cells treated with prostaglandin E(2). Eur J Obstet Gynecol Reprod Biol 2008; 138:83-8.
16. Peddada S D, Laughlin S K, Miner K, et al. Growth of uterine leiomyomata among premenopausal black and white women. Proc Natl Acad Sci USA 2008; 105:19887-92.
17. Thonneau P, Almont T, de La Rochebrochard E, Maria B. Risk factors for IUD failure: results of a large multicentre case-control study. Hum Reprod 2006; 21:2612-6.
18. Misro M M, Chaki S P, Chandra M, Maheshwari A, Nandan D. Release of copper from CuT 380A co-incubated with human semen and its effect on sperm function in vitro. Indian J Physiol Pharmacol 2008; 52:267-73.
19. Wu X, Blanck A, Olovsson M, Moller B, Favini R, Lindblom B. Apoptosis, cellular proliferation and expression of p53 in human uterine leiomyomas and myometrium during the menstrual cycle and after menopause. Acta Obstet Gynecol Scand 2000; 79:397-404.
20. Wildemeersch D. New frameless and framed intrauterine devices and systems—an overview. Contraception 2007; 75:S82-92.
21. Altenburger R, Rohr U D, Kissel T. Rate control in transdermal beta-estradiol reservoir membrane systems: the role of membrane and adhesive layer. Pharm Res 1998; 15:1238-43.
22. Zhang X, Yuan H, Deng L, Hu F, Ma J, Lin J. Evaluation of the efficacy of a danazol-loaded intrauterine contraceptive device on adenomyosis in an ICR mouse model. Hum Reprod 2008; 23:2024-30.
23. Gurates B, Parmaksiz C, Kilic G, Celik H, Kumru S, Simsek M. Treatment of symptomatic uterine leiomyoma with letrozole. Reprod Biomed Online 2008; 17:569-74.
24. Hilario S G, Bozzini N, Borsari R, Baracat E C. Action of aromatase inhibitor for treatment of uterine leiomyoma in perimenopausal patients. Fertil Steril 2009; 91:240-3.
25. Cobellis L, Razzi S, Fava A, Severi F M, Igarashi M, Petraglia F. A danazol-loaded intrauterine device decreases dysmenorrhea, pelvic pain, and dyspareunia associated with endometriosis. Fertil Steril 2004; 82:239-40.
26. Boeddinghaus I M, Dowsett M. Comparative clinical pharmacology and pharmacokinetic interactions of aromatase inhibitors. J Steroid Biochem Mol Biol 2001; 79:85-91.
27. Altenburger R, Rohr U D, Kissel T. Rate control in transdermal beta-estradiol reservoir membrane systems: the role of membrane and adhesive layer. Pharm Res 1998; 15:1238-43.
28. Wildemeersch D. New frameless and framed intrauterine devices and systems—an overview. Contraception 2007; 75:S82-92.
29. Li J, Suo J, Huang X, Jia L. Study on a novel copper-containing composite for contraception. Contraception 2009; 79:439-44.

1. A method comprising delivering an AI locally to the uterus of a patient in need of such treatment.

2. The method of claim 1, wherein the delivery rate is an extended or controlled release rate.

3. The method of claim 1, wherein the AI is selected from the group consisting of Exemestane (Aromasin®), Anastrozole (Arimidex®), Letrozole (Femara®), Aminoglutethimide (Cytadren®), Rogletimide (an analog of aminoglutethimide), Formestane (Lentaron®), Fadrozole (Afema®), Testolactone (Teslac®), Vorozole (Rivizor®), 4-androstene-3,6,17-trione (“6-OXO”), 1,4,6-androstatrien-3,17-dione (ATD), and 4-hydroxyandrostenedione (4-OHA).

4. The method of claim 1, further comprising co-administering an additional agent with the AI.

5. The method of claim 4, wherein the additional agent is a contraceptive.

6. A method of treating fibroids in a patient in need of such treatment comprising delivering an AI locally to the uterus.

7. The method of claim 6, wherein the delivery rate is an extended or controlled release rate.

8. The method of claim 6, wherein the AI is selected from the group consisting of Exemestane (Aromasin®), Anastrozole (Arimidex®), Letrozole (Femara®), Aminoglutethimide (Cytadren®), Rogletimide (an analog of aminoglutethimide), Formestane (Lentaron®), Fadrozole (Afema®), Testolactone (Teslac®), Vorozole (Rivizor®), 4-androstene-3,6,17-trione (“6-OXO”), 1,4,6-androstatrien-3,17-dione (ATD), and 4-hydroxyandrostenedione (4-OHA).

9. The method of claim 6, further comprising co-administering an additional agent with the AI.

10. The method of claim 9, wherein the additional agent is a contraceptive.

11. A method of preventing fibroids in a patient in need of such treatment comprising delivering an AI locally to the uterus.

12. The method of claim 11, wherein the delivery rate is an extended or controlled release rate.

13. The method of claim 11, wherein the AI is Exemestane (Aromasin®), Anastrozole (Arimidex®), Letrozole (Femara®), Aminoglutethimide (Cytadren®), Rogletimide (an analog of aminoglutethimide), Formestane (Lentaron®), Fadrozole (Afema®), Testolactone (Teslac®), Vorozole (Rivizor®), 4-androstene-3,6,17-trione (“6-OXO”), 1,4,6-androstatrien-3,17-dione (ATD), and 4-hydroxyandrostenedione (4-OHA).

14. The method of claim 11, further comprising co-administering an additional agent with the AI.

15. The method of claim 14, wherein the additional agent is a contraceptive.

16. A device for administering an AI locally to a uterus, comprising an IUD, and a reservoir for storing and dispensing the AI.

17. The device of claim 16, wherein the device further comprises an additional reservoir for storing and dispensing an additional active agent.

18. The device of claim 16, wherein the AI is Exemestane (Aromasin®), Anastrozole (Arimidex®), Letrozole (Femara®), Aminoglutethimide (Cytadren®), Rogletimide (an analog of aminoglutethimide), Formestane (Lentaron®), Fadrozole (Afema®), Testolactone (Teslac®), Vorozole (Rivizor®), 4-androstene-3,6,17-trione (“6-OXO”), 1,4,6-androstatrien-3,17-dione (ATD), and 4-hydroxyandrostenedione (4-OHA).

19. The device of claim 17, wherein the additional active agent is a contraceptive.

20. A formulation comprising an AI, and additional agent or agents which control the release rate of the AI.

21. The formulation of claim 20, further comprising an additional active agent.

22. The formulation of claim 21, wherein the additional active agent is a contraceptive.
